(19) United States
(12) Patent Application Publication
(10) Pub. No.: US 2025/0387037 A1
(43) Pub. Date: Dec. 25, 2025

(54) NON-CONTACT REAL-TIME MONITORING SYSTEM OF PHYSIOLOGICAL SIGNS BASED ON MILLIMETER-WAVE RADAR

(71) Applicant: Beijing University of Posts and Telecommunications, Beijing (CN)

(72) Inventors: Shuai Zhao, Beijing (CN); Shengjie Li, Beijing (CN); Jiapeng Tang, Beijing (CN); Bo Cheng, Beijing (CN); Junliang Chen, Beijing (CN)

(21) Appl. No.: 19/242,104

(22) Filed: Jun. 18, 2025

(30) Foreign Application Priority Data
Jun. 21, 2024 (CN) 2024108084206

(51) Int. Cl. A61B 5/05; A61B 5/00; A61B 5/08

(52) U.S. Cl. CPC A61B 5/05; A61B 5/08; A61B 5/7203; A61B 5/7239; A61B 5/725; A61B 5/7257; A61B 5/7271

(57) ABSTRACT

A non-contact real-time monitoring system of physiological signs based on millimeter-wave radar includes a millimeter-wave radar and multiple modules for processing radar signals. The millimeter-wave radar is configured to continuously transmit electromagnetic wave signals and simultaneously receive echo signals, perform frequency mixing processing on the echo signals to obtain an intermediate frequency signal, and process the intermediate frequency signal to obtain a radar four-dimensional data matrix. Human body physiological signs are monitored by analyzing body thoracic cavity micro-motion information in signals through the modules; a target echo is processed by adopting a constant false alarm rate detection algorithm, and invalid signals are filtered. A self-adaptive range cell selection algorithm based on short-time stability of respiratory signals is adopted to capture radar echoes reflecting physiological movement. Mixed human body physiological sign signals are processed by using a VMD algorithm, and key parameters in VMD are optimized by using a GWO algorithm.

### CROSS-REFERENCE TO RELATED APPLICATION

This application claims priority to Chinese Patent Application No. 202410808420.6, filed on Jun. 21, 2024, which is herein incorporated by reference in its entirety.

### TECHNICAL FIELD

The disclosure relates to the technical field of non-contact real-time physiological sign monitoring, and more particularly to a non-contact real-time monitoring system of physiological signs based on millimeter-wave radar.

### BACKGROUND

With the continuous development of society and economy, people pay more and more attention to their personal health. This trend reflects pursuit of high-quality life and concern for health of people. Moreover, with acceleration of modern life, increase of work pressure and increasingly serious problem of population aging, people of different ages have more urgent needs for scientific disease prevention and daily health monitoring. In the medical field, breathing, body temperature, pulse and blood pressure are called four major physiological signs. They are pillars for maintaining normal activities of a body and can directly reflect a state of human life activities.

Most common physiological sign monitoring methods are contact-based, such as electrocardiograms and blood oximeters. Such contact-based devices usually require professional medical staff to operate and are not friendly to some special patients, such as burn patients, patients with mental problems, and infants. Therefore, it is of great significance to explore a non-contact monitoring method of physiological signs for solving a problem of patients suffering from instrument constraints and realizing long-term monitoring and remote sensing.

Specifically, millimeter-wave radar has significant advantages and potential in the field of non-contact detection of the physiological signs. The millimeter-wave radar refers to a radar that works in a millimeter-wave band (a frequency band of 30 megahertz (GHz) to 300 GHz, with a wavelength of generally 1 millimeter (mm) to 10 mm). Human tissues such as skin, muscles, and bones have a certain reflectivity to millimeter-waves, which means that when the millimeter-waves come into contact with or pass through the human body, they will be reflected and generate reflected signals. Advantages of the millimeter-wave radar is that it can penetrate materials and clothing, and is less affected by environmental factors such as smoke, temperature and humidity. This means that it can achieve all-weather and all-day monitoring of the physiological signs, and can effectively avoid a risk of privacy leakage, with a high degree of reliability and privacy.

Although the millimeter-wave radar has many advantages in monitoring human physiological signs, its application still faces some challenges. (1) Vibration caused by heartbeat is significantly weaker than vibration caused by breathing, which causes modulation of a heartbeat signal to the radar to be submerged by the latter, making it much more difficult to estimate the heartbeat signal than a breathing signal. (2) The signal received by the millimeter-wave radar may be affected by many factors, such as changes in human posture, irregular changes in breathing and heartbeat frequencies, and environmental noise. Therefore, accurate and reliable signal processing of the received signal is a huge challenge. (3) When separating the breathing and heartbeat signals, it must be able to adapt to changes in signal strength, eliminate noise interference, and have sufficient adaptability to handle signal changes under different human structures and physiological conditions.

### SUMMARY

In order to achieve the above problems, the disclosure provides a non-contact real-time monitoring system of physiological signs based on millimeter-wave radar, to achieve non-contact real-time monitoring of the physiological signs.

In order to achieve the above purpose, the disclosure provides a non-contact real-time monitoring system of physiological signs based on millimeter-wave radar, which adopts the following technical solutions.

A non-contact real-time monitoring system of physiological signs based on millimeter-wave radar includes a millimeter-wave radar, a clutter suppression module, a target identification module, a range cell determination module, a phase signal extraction module, a signal decomposition module, and a physiological sign signal estimation module.

The millimeter-wave radar is configured to continuously transmit electromagnetic wave signals and simultaneously receive echo signals, perform frequency mixing processing on the echo signals to obtain an intermediate frequency signal, and process the intermediate frequency signal to obtain a radar four-dimensional data matrix Y∈ [symbol] ^{M×N×L×K}. Specifically, M represents an acquisition frame rate; N represents a number of pulses per frame; L represents a number of channels; K represents a number of sampling points; and [symbol] represents a complex domain.

The clutter suppression module is configured to perform fast Fourier transform (i.e., first Fourier transform) on the radar four-dimensional data matrix to obtain a radar signal after fast Fourier transform, and perform static clutter suppression on the radar signal after fast Fourier transform to obtain a radar signal after static clutter suppression.

The target identification module is configured to perform non-coherent pulse integration on the radar signal after static clutter suppression to obtain range spectrum data, and identify a target according to the range spectrum data.

The range cell determination module is configured to adaptively select a target range cell based on short-term stationarity of a respiratory signal in response to the target identification module identifying the target.

The phase signal extraction module is configured to extract phase information of the target range cell to obtain a phase signal.

The signal decomposition module is configured to process the phase signal by using a variational mode decomposition algorithm to decompose the phase signal into a linear combination of multiple modes, and determine a weight and a center frequency of each mode through an optimization problem.

The physiological sign signal estimation module is configured to perform Fourier transform (i.e., second Fourier transform) on the linear combination of the multiple modes to obtain a spectrum for each mode of the linear combination of the multiple modes, search for spectral peaks from the spectrum to perform frequency estimation, and screen a component satisfying characteristics of the physiological signs; and use a component with a minimum envelope entropy as an estimating result of a target physiological sign signal in response to multiple components existed in an interval.

In an exemplary embodiment, each of the clutter suppression module, the target identification module, the range cell determination module, the phase signal extraction module, the signal decomposition module, and the physiological sign signal estimation module is embodied by at least one processor and at least one memory coupled to the at least one processor, and the at least one memory stores programs executable by the at least one processor.

In an embodiment, each electromagnetic wave signal continuously transmitted by the millimeter-wave radar is expressed as follows:

$${S}_{T} ( t ) = {A}_{T} cos ( 2 π {f}_{c} t + \frac{π B}{{T}_{c}} {t}^{2} + φ ( t ) )$$

where S_{T}(t) represents the electromagnetic wave signal continuously transmitted by the millimeter-wave radar, f_{c} represents an initial frequency, B represents a frequency modulation bandwidth, T_{c} represents a frequency modulation cycle, cos represents a cosine function, π represents a pi, A_{T} represents an amplitude, t represents a time, and φ(t) represents an initial phase.

In an embodiment, the intermediate frequency signal is expressed as follows:

$${S}_{IF} ( t ) = {A}_{T} {e}^{4 π \frac{BR ( t )}{c {T}_{c}} + 4 π \frac{{f}_{c} R ( t )}{c}}$$

where S_{IF}(t) represents the intermediate frequency signal, f_{c} represents an initial frequency, B represents a frequency modulation bandwidth, T_{c} represents a frequency modulation cycle, e represents a natural constant, π represents a pi, R(t) represents a function of distance changing over time, A_{T} represents an amplitude of the intermediate frequency signal, t represents a time, and c represents a speed of light.

In an embodiment, the clutter suppression module is further configured to estimate a degree of static clutter interference in environment by calculating an average of signals on each of range cells in a certain period, and a formula of the estimate is expressed as follows:

$$C [ n ] = \frac{1}{N} {\sum }_{m = 1}^{N} R [ n , m ]$$

where C[n] represents an average clutter estimate in a time window, m represents a range cell, and R[n,m] represents a radar echo signal strength received at a time point n and the range cell m.

In an embodiment, the target identification module is further configured to:
classify the range spectrum data into multiple windows each including data to be processed, select one of range cells from each of the multiple windows as a center one by one, with Nᵣ/2 range cells in front and behind, and exclude a middle protection range cell to form a reference window Wᵢ;
sort values in the reference window Wᵢ in an ascending order;
calculate a threshold multiplier T, and select a kₒₛ^{th} largest value in the reference window Wᵢ as a local estimated background noise level based on a false alarm probability P_{FA} and a window size Nᵣ; and
calculate a detection threshold, in response to a test cell of one of the range cells value greater than the detection threshold, mark one of the range cells as the target; or in response to a test cell value of one of the range cells smaller than or equal to the detection threshold, and mark the one of the range cells as a non-target.

In an embodiment, the range cell determination module is further configured to:
determine a range cell m₁ with a maximum reflected power by the range spectrum data after non-coherent pulse integration;
select two range cells from a left side and a right side of the range cell m₁ respectively to form a candidate set R={m₁−2,m₁−1,m₁,m₁+1,m₁+2};
extract phase information ϕ₁ and ϕ₂ of adjacent pulse signals x₁ and x₂ in a same frame according to the range cells in the candidate set respectively;
extract respiratory signal components B₁ and B₂ in the phase information ϕ₁ and ϕ₂ through a [0.1 Hz, 1 Hz] band-pass filter respectively;
calculate Pearson correlation coefficients for the respiratory signal components B₁ and B₂, respectively; and
select a range cell R* with a maximum Pearson correlation coefficient between the respiratory signal component B₁ and B₂ as follows:

$${R}^{*} = arg max PCC ( {B}_{1}^{r} , {B}_{2}^{r} ) r ∈ R$$

where argmax represents a maximum operation, PCC represents the Pearson correlation coefficient,

$${B}_{1}^{r}$$

represents a first pulse respiratory component,

$${B}_{2}^{r}$$

represents a second pulse respiratory component, r represents an element in the candidate set of the range cells, and R represents the candidate set of the range cells.

In an embodiment, the range cell determination module is further configured to calculate the Pearson correlation coefficients for the respiratory signal components B₁ and B₂ through the following formula:

$$PCC ( X , Y ) = \frac{{\sum }_{i = 1}^{len} ( {X}_{i} - \overline{X} ) ( {Y}_{i} - \overline{Y} )}{\sqrt{{\sum }_{i = 1}^{len} {( {X}_{i} - \overline{X} )}^{2}} \sqrt{{\sum }_{i = 1}^{len} {( {Y}_{i} - \overline{Y} )}^{2}}}$$

where i represents an index variable, Xᵢ represents a value of a variable X corresponding to an i^{th} observation value, Yᵢ represents a value of a variable Y corresponding to the i^{th} observation value, len represents a number of the observation values, X̅ represents a mean value of the variable X, and Y̅ represents a mean value of the variable Y.

In an embodiment, the phase signal extraction module is further configured to:
calculate an initial phase φ[t] of the target through the following formula:

$$φ [ t ] = Arctan ( \frac{sin ( φ [ t ] )}{cos ( φ [ t ] )} ) - \frac{π}{2} < φ [ t ] < \frac{π}{2}$$

perform phase unwrapping to obtain an unwrapped signal sample by sample, determine whether a phase difference between adjacent samples φ[t] and φ[t+1] of the target range cell exceeds a threshold π, in response to the phase difference greater than π, subtract 2π; and in response to the phase difference not greater than π, add 2π;
perform first-order differential processing on the unwrapped signal to extract relative phase variation between the adjacent samples to thereby obtain a signal after first-order differential processing; and
perform smooth filtering processing on the signal after first-order differential processing.

In an embodiment, the signal decomposition module is further configured to optimize a number of the multiple modes and a penalty coefficient of the variational mode decomposition algorithm by using a gray wolf optimization algorithm, including:
using the number of the multiple modes and the penalty coefficient as optimization variables, and using minimization of envelope entropy as a fitness function;
performing, according to a position of a gray wolf and using corresponding variational mode decomposition (VMD) parameters, signal decomposition on the phase signal to obtain a set of modes, and calculating a minimum envelope entropy of the set of modes as a fitness value;
updating the position of the gray wolf according to the fitness value and updating rules; and
outputting a current number of the multiple modes and a current penalty coefficient as a target solution in response to satisfying a termination condition.

In an embodiment, the signal decomposition module is further configured to:
construct an analysis signal of each of mode functions through Hilbert transform, where each of the mode functions corresponds to one of the multiple modes; calculate a single-sided spectrum of the analysis signal, move the single-sided spectrum to a baseband, calculate a L² norm of a gradient square of a demodulated signal to estimate a bandwidth of the demodulated signal, and minimize a sum of spectral widths of all of the mode functions by constructing a constrained variational problem, where the constrained variational problem is expressed as follows:

$${min}_{\{ {μ}_{k} \} , \{ {ω}_{k} \}} ( {\sum }_{k} {‖ \partial t [ ( δ ( t ) + \frac{j}{π t} ) * {μ}_{k} ( t ) ] \cdot {e}^{- j {ω}_{k} t} ‖}_{2}^{2} \}$$

$$s . t . {\sum }_{k = 1}^{K} {μ}_{k} ( t ) = f ( t )$$

where min_{{μ}_{ₖ}_{}, {ω}_{ₖ}_{}} represents a target of optimization, namely a solution for minimizing the sum of the spectral widths of all mode functions; μₖ represents a set of the mode functions; ωₖ represents a set of frequency parameters, configured to describe frequency characteristics of each of the mode functions; k represents a serial number of each of the mode functions, and K represents a total number of the mode functions; t represents a time variable, configured to describe variation of signals over time; δ(t) represents a unit pulse function; j represents an imaginary unit; μₖ(t) represents a specific form of each of the mode functions; and f(t) represents an input signal, namely a signal to be decomposed;
convert the constrained variational problem into an unconstrained optimization problem by introducing the penalty coefficient α and a Lagrange multiplier λ as follows:

$$ℓ ( \{ {μ}_{k} \} , \{ {ω}_{k} \} , λ ) = α {\sum }_{k} {‖ \partial t [ ( δ ( t ) + \frac{j}{π t} ) * {μ}_{k} ( t ) ] \cdot {e}^{- j {ω}_{k} t} ‖}_{2}^{2} + {‖ f ( t ) - {\sum }_{k} {μ}_{k} ( t ) ‖}_{2}^{2} + 〈 λ ( t ) , f ( t ) - {\sum }_{k} {μ}_{k} ( t ) 〉$$

where [symbol] represents a target function for optimization, comprising the penalty coefficient and the Lagrange multiplier; λ(t) represents the Lagrange multiplier, configured to ensure that a sum of the input signal and the mode functions is equal to an original signal; f(t)−Σₖμₖ(t) represents a residual of the sum of the input signal and the mode functions, representing whether the input signal minus a combination of the all mode functions is equal to zero; and λ(t),f(t)−Σₖμₖ(t) represents an inner product of the Lagrange multiplier and the residual, configured to ensure satisfaction of constraint conditions; and
perform iterative solution by using an alternating direction method of multipliers, and divide the unconstrained optimization problem into two sub-optimization problems to solve an overall optimal solution, in response to satisfying a convergence condition or reaching a maximum number of iterations, stop the iteration solution, and complete the signal decomposition.

The disclosure at least has the following beneficial effects.

The disclosure utilizes the millimeter wave radar technology to realize real-time monitoring of human physiological signs indoors. Through a combination of multiple modules, the physiological signals such as chest micro-movements can be effectively extracted, and then physiological sign parameters can be accurately estimated.

### BRIEF DESCRIPTION OF DRAWINGS

In order to more clearly illustrate specific embodiments of the disclosure or technical solutions in the related art, drawings required for descriptions of the specific embodiments or the related art will be briefly introduced below. In all the drawings, similar elements or parts are generally identified by similar reference signs. In the drawings, the elements or parts are not necessarily drawn according to an actual scale.

FIG. 1 illustrates a schematic structural diagram of non-contact real-time monitoring system of physiological signs based on millimeter-wave radar according to an embodiment of the disclosure.

FIG. 2 illustrates a schematic diagram of a working principle of the non-contact real-time monitoring system of physiological signs based on millimeter-wave radar according to an embodiment of the disclosure.

FIG. 3 illustrates a workflow diagram of the non-contact real-time monitoring system of physiological signs based on millimeter-wave radar according to an embodiment of the disclosure.

FIG. 4 illustrates a distance spectrogram after static clutter suppression according to an embodiment of the disclosure.

FIG. 5 illustrates a schematic diagram of a target detection effect according to an embodiment of the disclosure.

FIG. 6 illustrates a schematic diagram of a smooth filtering effect according to an embodiment of the disclosure.

FIG. 7 illustrates a schematic diagram of a variational mode decomposition result according to an embodiment of the disclosure.

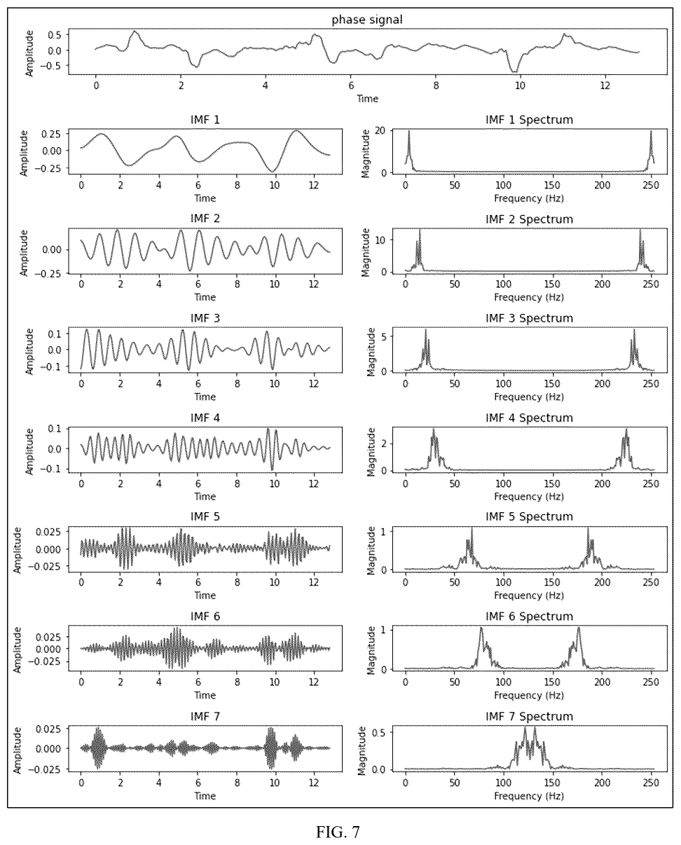

### DETAILED DESCRIPTION OF EMBODIMENTS

Embodiments of the disclosure are illustrated by specific examples below, and those skilled in the art can easily understand other advantages and effects of the disclosure from contents disclosed in this specification. The disclosure can also be implemented or applied through other different specific embodiments, and details in this specification can also be modified or changed in various ways based on different viewpoints and applications without departing from a spirit of the disclosure. It should be noted that the following embodiments and features in the embodiments can be combined with each other without conflict.

In description of the disclosure, unless otherwise specified, “plurality” means two or more than two. Orientations or positional relationships indicated by terms “upper”, “lower”, “left”, “right”, “inner”, “outer”, “front end”, “rear end”, “head”, “tail”, and the like are based on orientations or positional relationships shown in the drawings, and are only for convenience of describing the disclosure and simplifying the description, and do not indicate or imply that a device or element referred to must have a specific orientation, be constructed and operate in a specific orientation, and therefore cannot be understood as limiting the disclosure. In addition, terms “first”, “second”, “third” and the like are only used for descriptive purposes and cannot be understood as indicating or implying relative importance.

In the description of the disclosure, it should be noted that, unless otherwise clearly specified and limited, terms “connected” and “connection” should be understood in a broad sense. For example, it can be a fixed connection, a detachable connection, or an integral connection; it can be a mechanical connection or an electrical connection; it can be a direct connection or an indirect connection through an intermediate medium. For those skilled in the art, specific meanings of the above terms in the disclosure can be understood according to specific circumstances.

The specific implementation of the disclosure is further described in detail below in conjunction with the drawings and the embodiments.

The embodiments of the disclosure provide an application scenario. In the application scenario, a target sits quietly at 0.75 meters (m) away from a millimeter-wave radar device. A hardware platform used in the experiment is a commercial millimeter-wave radar sensor IWR6843 produced by Texas Instruments, which has a sweep bandwidth of 4 GHZ and a corresponding distance resolution of 3.75 centimeters (cm). A single transmitting antenna is used in waveform configuration to continuously send electromagnetic waves at a frame rate of 20 hertz (Hz). According to a Nyquist sampling theorem, a sampling frequency must be higher than twice the highest heart rate in a physiological signal to meet extraction requirements of physiological sign signals. Each frame signal includes two Chirps, and a frame period is 50 milliseconds (ms). In each Chirp, the radar performs 256 analog-to-digital converter (ADC) samplings, and a duration of the Chirp is 50 microseconds (μs). During the experiment, the subjects wore a finger-clip pulse oximeter to record heartbeat data as a reference.

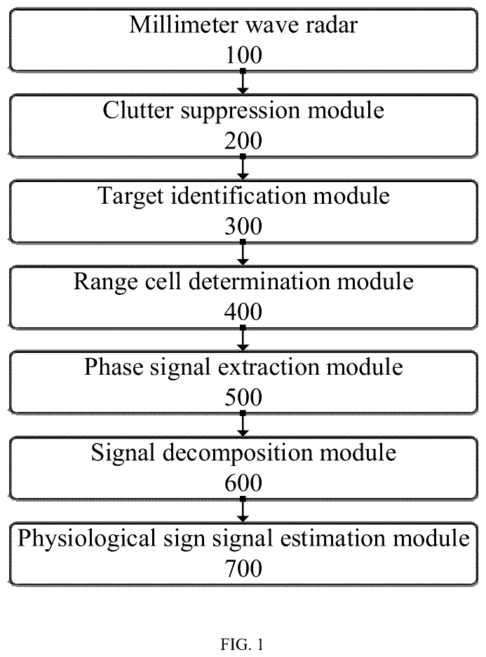

Based on the above application scenario, the embodiments of the disclosure provide a non-contact real-time monitoring system of physiological signs based on millimeter-wave radar, as shown in FIG. 1, the system includes a millimeter-wave radar 100, a clutter suppression module 200, a target identification module 300, a range cell determination module 400, a phase signal extraction module 500, a signal decomposition module 600, and a physiological sign signal estimation module 700 in signal connection in sequence.

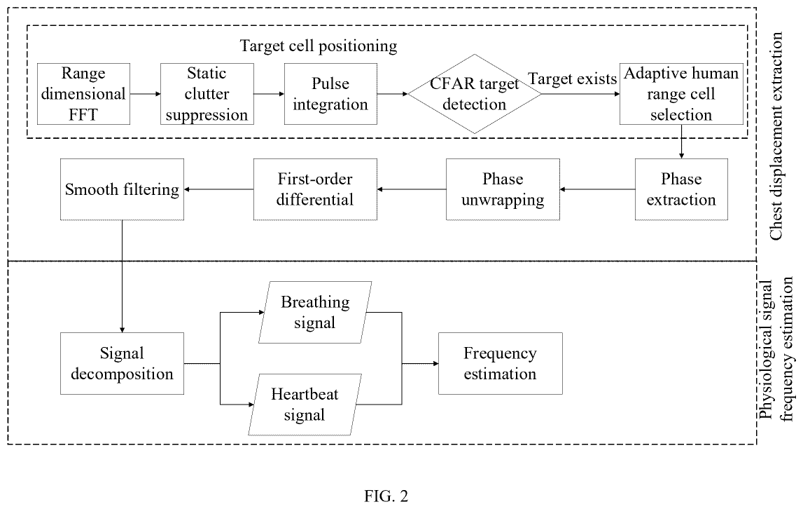

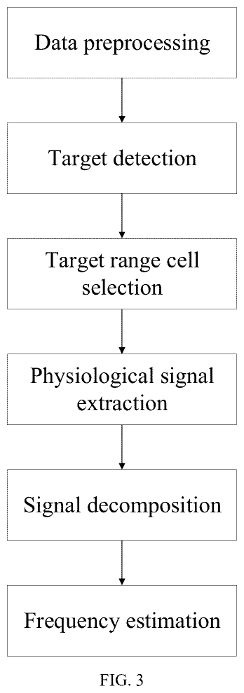

Working principles of the millimeter-wave radar 100 and other modules included by the system will be introduced below in conjunction with a working principle shown in FIG. 2 and a workflow shown in FIG. 3.

The millimeter-wave radar 100 is configured to continuously transmit electromagnetic wave signals and simultaneously receive echo signals, perform frequency mixing processing on the echo signals to obtain an intermediate frequency signal, and process the intermediate frequency signal to obtain a radar four-dimensional data matrix Y∈ [symbol] ^{M×N×L×K}. Specifically, M represents an acquisition frame rate; N represents a number of pulses per frame; L represents a number of channels; K represents a number of sampling points; and [symbol] represents a complex domain.

In some embodiments, each electromagnetic wave signal continuously transmitted by the millimeter-wave radar 100 is expressed as follows:

$${S}_{T} ( t ) = {A}_{T} cos ( 2 π {f}_{c} t + \frac{π B}{{T}_{c}} {t}^{2} + φ ( t ) )$$

where S_{T}(t) represents the electromagnetic wave signal continuously transmitted by the millimeter-wave radar, f_{c} represents an initial frequency, B represents a frequency modulation bandwidth, T_{c} represents a frequency modulation cycle, cos represents a cosine function, π represents a pi, A_{T} represents an amplitude, t represents a time, and φ(t) represents an initial phase.

After the millimeter-wave radar 100 receives the echo signals, the echo signals and local oscillator signals are processed in a mixer to obtain the intermediate frequency signal. An essence of frequency mixing is spectrum shifting, which uses non-linear elements to multiply two signals (i.e., the echo signals and the local oscillator signals) to achieve frequency conversion and processing. Through the frequency mixing process, the high-frequency signal can be converted into the intermediate frequency signal, which is convenient for subsequent signal processing and demodulation. The intermediate frequency signal is expressed as follows:

$${S}_{IF} ( t ) = {A}_{T} {e}^{4 π \frac{BR ( t )}{c {T}_{c}} + 4 π \frac{{f}_{c} R ( t )}{c}}$$

where S_{IF}(t) represents the intermediate frequency signal, f_{c} represents an initial frequency, B represents a frequency modulation bandwidth, T, represents a frequency modulation cycle, e represents a natural constant, π represents a pi, R(t) represents a function of distance changing over time, A_{T} represents an amplitude of the intermediate frequency signal, t represents a time, and c represents a speed of light.

Breathing and heartbeat are main manifestations of physiological activities, which are quasi-periodic motions. In order to simplify the processing, a motion law of human breathing and heartbeat is approximated as a sinusoidal signal in the embodiment. Therefore, a chest displacement is expressed as follows:

$$R ( t ) = {A}_{b} sin ( 2 π {f}_{b} t ) + {A}_{h} ( 2 π {f}_{h} t ) + {η}_{t}$$

where A_{b} represents an amplitude of a breathing signal, and Aₕ represents an amplitude of a heartbeat signal; and f_{b} represents a breathing frequency, fₕ represents a heartbeat frequency, and nₜ represents random noise.

The clutter suppression module 200 is configured to perform fast Fourier transform (i.e., first Fourier transform) on a radar original signal (i.e., the radar four-dimensional data matrix) along a fast time dimension to obtain a radar signal after fast Fourier transform, and perform static clutter suppression on the radar signal after fast Fourier transform to obtain a radar signal after static clutter suppression by a mean cancellation technique.

Specifically, the clutter suppression module 200 uses the mean cancellation technique to perform the static clutter suppression, to eliminate interference of direct current (DC) components and static impurities in the radar signal after fast Fourier transform. An average of signals on each range cell in a certain period is calculated to estimate a degree of static clutter interference in environment, and a formula is expressed as follows:

$$C [ n ] = \frac{1}{N} {\sum }_{m = 1}^{N} R [ n , m ]$$

where C[n] represents an average clutter estimate in a time window, m represents a range cell, N represents a total number of the range cells, and R[n,m] represents a radar echo signal strength received at a time point n and the range cell m.

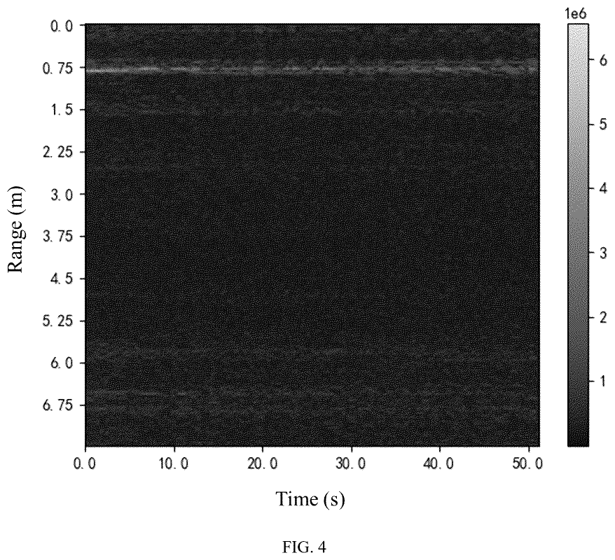

The target identification module 300 is configured to perform non-coherent pulse integration on the radar signal after static clutter suppression of the clutter suppression module 200 to obtain range spectrum data, which is shown in FIG. 4, and process the range spectrum data after non-coherent pulse integration by using an ordered statistics constant false alarm rate (OS-CFAR) detection algorithm.

In some embodiments, after performing the static clutter suppression on the radar signal, the non-coherent pulse integration is performed on the radar signal through the following formula:

$${R}_{acc} [ n ] = {\sum }_{k = 1}^{N} \vert R [ n , k ] \vert$$

where R_{acc}[n] represents a radar echo signal strength integrated at the time point n, k represents a range cell, n represents a time point, and R[n,k] represents a radar echo signal strength received at the time point n and the range cell k.

In some embodiments, the target identification module 300 is further configured to process the range spectrum data after non-coherent pulse integration by following steps 3.1-3.4.

In step 3.1, the range spectrum data is classified into multiple windows, and each window includes data to be processed. A range cell is selected from each window as a center one by one, with Nᵣ/2 range cells in front and behind, and a middle protection range cell is excluded to form a reference window Wᵢ.

In step 3.2, values in the reference window Wᵢ are sorted in an ascending order.

In step 3.3, a threshold multiplier T is calculated, and a kₒₛ^{th} largest value in the reference window Wᵢ is selected as a local estimated background noise level based on a false alarm probability P_{FA} and a window size Nᵣ.

In step 3.4, a detection threshold is calculated. In response to a test cell value of a range cell greater than the detection threshold, the range cell is marked as the target; otherwise, the range cell is marked as a non-target.

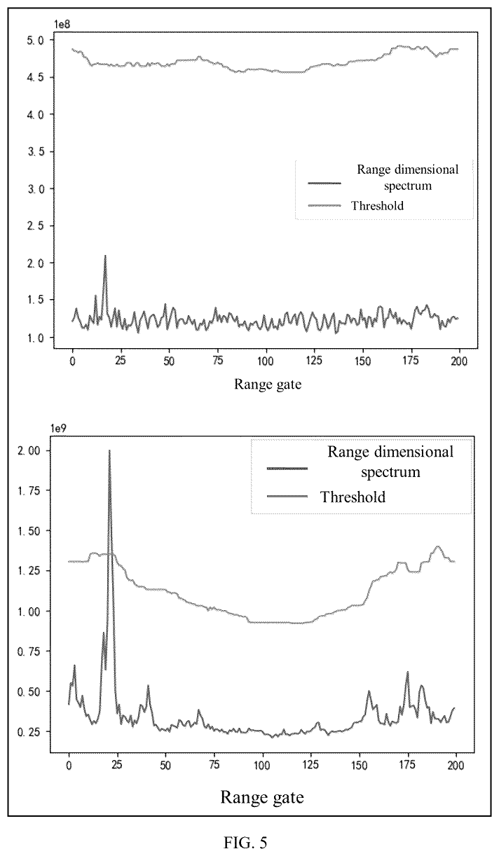

A target detection result achieved by the target identification module 300 is shown in FIG. 5.

The range cell determination module 400 is configured to adaptively select a target range cell based on short-term stationarity of a respiratory signal, so that the extracted phase information can better reflect the physiological signs of the human body.

In some embodiments, the range cell determination module 400 is further configured to adaptively select the target range cell through the following steps 4.1-4.6.

In step 4.1, the processing of the echo signals is performed, and a range cell m₁ with a maximum reflected power is determined by the range spectrum data after non-coherent pulse integration.

In step 4.2, two range cells are selected from a left side and a right side of the range cell m₁ respectively to form a candidate set R={m₁−2,m₁−1,m₁,m₁+1,m₁+2}.

In step 4.3, phase information ϕ₁ and ϕ₂ of adjacent pulse signals x₁ and x₂ in a same frame are extracted according to the range cells in the candidate set respectively.

In step 4.4, respiratory signal components B₁ and B₂ in the phase information ϕ₁ and ϕ₂ are extracted through a [0.1 Hz, 1 Hz] band-pass filter respectively.

In step 4.5, Pearson correlation coefficients for the respiratory signal components B₁ and B₂ is calculated respectively as follows:

$$PCC ( X , Y ) = \frac{{\sum }_{i = 1}^{l e n} ( {X}_{i} - \overline{X} ) ( {Y}_{i} - \overline{Y} )}{\sqrt{{\sum }_{i = 1}^{l e n} {( {X}_{i} - \overline{X} )}^{2}} \sqrt{{\sum }_{i = 1}^{l e n} {( {Y}_{i} - \overline{Y} )}^{2}}}$$

where i represents an index variable, Xᵢ represents a value of a variable X corresponding to an i^{th} observation value, Yᵢ represents a value of a variable Y corresponding to the i^{th} observation value, len represents a number of the observation values, X̅ represents a mean value of the variable X, and Y̅ represents a mean value of the variable Y.

In step 4.6, a range cell R* with a maximum Pearson correlation coefficient between the respiratory signal component B₁ and B₂ is selected as follows:

$${R}^{*} = argmax PCC ( {B}_{1}^{r} , {B}_{2}^{r} ) r ∈ R$$

where argmax represents a maximum operation, PCC represents the Pearson correlation coefficient,

$${B}_{1}^{r}$$

represents a first pulse respiratory component,

$${B}_{2}^{r}$$

represents a second pulse respiratory component, r represents an element in the candidate set of the range cells, and R represents the candidate set of the range cells.

The phase signal extraction module 500 is configured to extract phase information of the range cell for analysis after determining the target range cell of the human body.

In some embodiments, the phase signal extraction module 500 is further configured to extract the phase information of the range cell for analysis through the following steps 5.1-5.4.

In step 5.1, phase extraction is performed. The range dimensional fast Fourier transform (FFT) is performed on the signal, an arctangent method is performed on a real part and an imaginary part of the obtained result to calculate an initial phase of the target as follows:

$$φ [ t ] = Arctan ( \frac{sin ( φ [ t ] )}{cos ( φ [ t ] )} ) - \frac{π}{2} < φ [ t ] < \frac{π}{2} .$$

In step 5.2, phase unwrapping is performed. The arctangent method can effectively recover a phase of the target signal in a linear range as long as the phase remains in an interval (−π/2, π/2). However, when the phase exceeds the interval, a phase wrapping problem occurs. The phase wrapping problem is caused by properties of a tangent function, which may lead to a one-to-many mapping relationship. The tangent function is a surjective function only in the interval (−π/2, π/2). When exceeding the interval, the tangent function will map multiple different independent variables to a same value. Therefore, phase unwrapping processing is required, and the specific process is as follows.

The phase unwrapping is performed sample by sample, whether a phase difference between adjacent samples φ[t] and φ[t+1] exceeds a threshold π is determined, when the phase difference greater than π, it indicates that a jump has occurred and compensation is required. In response to the phase difference greater than π, subtract 2π; and in response to the phase difference not greater than π, add 2π.

In step 5.3, first-order differential processing is performed. The first-order differential processing is performed on the unwrapped signal to extract relative phase variation between the adjacent samples without being affected by an absolute phase value. In this way, even if the signal drifts, the phase difference can still accurately reflect the relative change of the target movement, reduce the impact of noise interference, and make the system more stable and reliable.

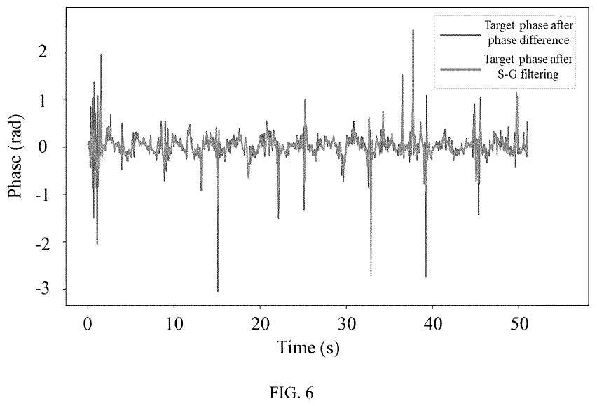

In step 5.4, smooth filtering processing is performed. The smooth filtering processing is performed on the signal after first-order differential processing to further eliminate the interference of environmental noise and random mutation signals. It is necessary to retain the overall characteristics and trends of the signal as much as possible while suppressing the noise. Therefore, a Savitzky-Golay (S-G) filtering algorithm is used to move windows in the signal and fit data in the window with a polynomial through a local polynomial fitting method, so as to achieve smooth processing of the signal. The effect diagram is shown in FIG. 6.

The signal decomposition module 600 is configured to process the above phase signal by using a variational mode decomposition (VMD) algorithm to decompose the phase signal into a linear combination of multiple modes, and determine a weight and a center frequency of each mode through an optimization problem, to thereby minimize energy leakage of the signal.

In some embodiments, the signal decomposition module 600 is further configured to optimize two key parameters, i.e., a number of modes and a penalty coefficient, of the VMD algorithm by using a gray wolf optimization (GWO) and through the following steps 6.1-6.4.

In step 6.1, related parameters of the GWO algorithm are initialized, the number of modes and the penalty coefficient of the VMD algorithm are used as optimization variables, and minimization of envelope entropy is used as a fitness function.

In step 6.2, signal decomposition is performed on the phase signal according to a position of a gray wolf and using corresponding VMD parameters to obtain a set of modes, and a minimum envelope entropy of the set of modes is calculated as a fitness value.

In step 6.3, the position of the gray wolf is updated according to the fitness value and updating rules, that is, optimal solution, sub-optimal solution, and general solution.

In step 6.4, a current number of modes and a current penalty coefficient are output as a target solution in response to satisfying a termination condition; otherwise, continue the iterative search.

In some embodiments, the signal decomposition module is further configured to as follows.

First, an analysis signal of each mode function is constructed through Hilbert transform, each mode function corresponds to one mode, a single-sided spectrum of the analysis signal is calculated, and the single-sided spectrum is moved to a baseband. A L² norm of a gradient square of a demodulated signal is calculated to estimate a bandwidth of the demodulated signal, and a sum of spectral widths of all mode functions is minimized by constructing a constrained variational problem. The constrained variational problem is expressed as follows:

$$\begin{matrix}{min}_{\{ {μ}_{k} \} , \{ {ω}_{k} \}} \{ {\sum }_{k} {‖ \partial t [ ( δ ( t ) + \frac{j}{π t} ) * {μ}_{k} ( t ) ] \cdot {e}^{- j {ω}_{k} t} ‖}_{2}^{2} \} \\ s . t . {\sum }_{k = 1}^{K} {μ}_{k} ( t ) = f ( t )\end{matrix}$$

where min_{{μ}_{ₖ}_{}, {ω}_{ₖ}_{}} represents a target of optimization, namely a solution for minimizing the sum of the spectral widths of all mode functions; μₖ represents a set of mode functions; dk represents a set of frequency parameters, configured to describe frequency characteristics of the mode functions; k represents a serial number of the mode function, representing different mode functions, and K represents a total number of the mode functions; t represents a time variable, configured to describe variation of signals over time; δ(t) represents a unit pulse function; j represents an imaginary unit; μₖ(t) represents a specific form of each mode function; and f(t) represents an input signal, namely a signal to be decomposed.

The penalty coefficient α and a Lagrange multiplier λ are introduced to convert the constrained variational problem into an unconstrained optimization problem as follows:

$$ℓ ( \{ {μ}_{k} \} , \{ {ω}_{k} \} , λ ) = α {\sum }_{k} {‖ \partial t [ ( δ ( t ) + \frac{j}{π t} ) * {μ}_{k} ( t ) ] \cdot {e}^{- j {ω}_{k} t} ‖}_{2}^{2} + {‖ f ( t ) - {\sum }_{k} {μ}_{k} ( t ) ‖}_{2}^{2} + 〈 λ ( t ) , f ( t ) - {\sum }_{k} {μ}_{k} ( t ) 〉$$

where [symbol] represents a target function for optimization, comprising the penalty coefficient and the Lagrange multiplier; λ(t) represents the Lagrange multiplier, configured to ensure that a sum of the input signal and the mode functions is equal to an original signal; f(t)−Σₖμₖ(t) represents a residual of the sum of the input signal and the mode functions, representing whether the input signal minus a combination of the all mode functions is equal to zero; and λ(t),f(t)−Σₖμₖ(t) represents an inner product of the Lagrange multiplier and the residual, configured to ensure satisfaction of constraint conditions.

An alternating direction method of multipliers (ADMM) is used to perform iteration solution, to divide the unconstrained optimization problem into two sub-optimization problems to solve an overall optimal solution. When a convergence condition is satisfied or a maximum iteration time is reached, iteration solution is stopped, and the signal decomposition is completed.

The physiological sign signal estimation module 700 is configured to perform Fourier transform (i.e., second Fourier transform) on the linear combination of the multiple modes to obtain a spectrum for each mode, search for spectral peaks from the spectrum to perform frequency estimation, and screen a component satisfying characteristics of the physiological signs, and use a component with a minimum envelope entropy as an estimating result of a target physiological sign signal in response to multiple components existed in an interval.

The non-contact real-time monitoring system of physiological signs based on millimeter-wave radar is applied to the application scenario mentioned in the embodiment. A reference value of a heart rate of the subject measured by a pulse oximeter is 68 to 72 times/minute. An estimation result of the heart rate obtained by the monitoring system of physiological signs based on millimeter-wave radar proposed in the embodiment is 70.2 times/minute, which is in line with the reference value range.

The above implementation modes are merely used to illustrate the disclosure, rather than limit the disclosure. Those skilled in the art can make various changes and modifications without departing from a spirit and a scope of the disclosure. Therefore, all equivalent technical solutions also belong to the scope of the disclosure. A scope of protection of the disclosure should be defined by claims.

1. A non-contact real-time monitoring system of physiological signs based on millimeter-wave radar, comprising:
a millimeter-wave radar, configured to continuously transmit electromagnetic wave signals and simultaneously receive echo signals, perform frequency mixing processing on the echo signals to obtain an intermediate frequency signal, and process the intermediate frequency signal to obtain a radar four-dimensional data matrix Y∈ [symbol] ^{M×N×L×K}, wherein M represents an acquisition frame rate; N represents a number of pulses per frame; L represents a number of channels; K represents a number of sampling points; and [symbol] represents a complex domain;
a clutter suppression module, configured to perform first Fourier transform on the radar four-dimensional data matrix to obtain a radar signal after first Fourier transform, and perform static clutter suppression on the radar signal after first Fourier transform to obtain a radar signal after static clutter suppression;
a target identification module, configured to perform non-coherent pulse integration on the radar signal after static clutter suppression to obtain range spectrum data, and identify a target according to the range spectrum data;
a range cell determination module, configured to adaptively select a target range cell based on short-term stationarity of a respiratory signal in response to the target identification module identifying the target;
a phase signal extraction module, configured to extract phase information of the target range cell to obtain a phase signal;
a signal decomposition module, configured to process the phase signal by using a variational mode decomposition algorithm to decompose the phase signal into a linear combination of a plurality of modes, and determine a weight and a center frequency of each of the plurality of modes through an optimization problem; and
a physiological sign signal estimation module, configured to perform second Fourier transform on the linear combination of the plurality of modes to obtain a spectrum for each mode of the linear combination of the plurality of modes, search for spectral peaks from the spectrum to perform frequency estimation, and screen a component satisfying characteristics of the physiological signs; and using a component with a minimum envelope entropy as an estimating result of a target physiological sign signal in response to a plurality of components existed in an interval;
wherein the signal decomposition module is further configured to optimize a number of the plurality of modes and a penalty coefficient of the variational mode decomposition algorithm by using a gray wolf optimization algorithm, comprising:
using the number of the plurality of modes and the penalty coefficient as optimization variables, and using minimization of envelope entropy as a fitness function;
performing, according to a position of a gray wolf and using corresponding variational mode decomposition (VMD) parameters, signal decomposition on the phase signal to obtain a set of modes, and calculating a minimum envelope entropy of the set of modes as a fitness value;
updating the position of the gray wolf according to the fitness value and updating rules; and
outputting a current number of the plurality of modes and a current penalty coefficient as a target solution in response to satisfying a termination condition;
wherein the signal decomposition module is further configured to:
construct an analysis signal of each of mode functions through Hilbert transform, wherein each of the mode functions corresponds to one of the plurality of modes; calculate a single-sided spectrum of the analysis signal, move the single-sided spectrum to a baseband, calculate a L² norm of a gradient square of a demodulated signal to estimate a bandwidth of the demodulated signal, and minimize a sum of spectral widths of all of the mode functions by constructing a constrained variational problem, wherein the constrained variational problem is expressed as follows:
$$\begin{matrix}{min}_{\{ {μ}_{k} \} , \{ {ω}_{k} \}} \{ {\sum }_{k} {‖ \partial t [ ( δ ( t ) + \frac{j}{π t} ) * {μ}_{k} ( t ) ] \cdot {e}^{- j {ω}_{k} t} ‖}_{2}^{2} \} \\ s . t . {\sum }_{k = 1}^{K} {μ}_{k} ( t ) = f ( t )\end{matrix}$$
wherein min_{{μ}_{ₖ}_{},{ω}_{ₖ}_{}} represents a target of optimization, namely a solution for minimizing the sum of the spectral widths of all of the mode functions; μₖ represents a set of mode functions; ωₖ represents a set of frequency parameters, configured to describe frequency characteristics of each of the mode functions; k represents a serial number of each of the mode functions, and K represents a total number of the mode functions; t represents a time variable, configured to describe variation of signals over time; δ(t) represents a unit pulse function; j represents an imaginary unit; μₖ(t) represents a specific form of each of the mode functions; and f(t) represents an input signal, namely a signal to be decomposed;
convert the constrained variational problem into an unconstrained optimization problem by introducing the penalty coefficient α and a Lagrange multiplier λ as follows:
$$ℓ ( \{ {μ}_{k} \} , \{ {ω}_{k} \} , λ ) = α {\sum }_{k} {‖ \partial t [ ( δ ( t ) + \frac{j}{π t} ) * {μ}_{k} ( t ) ] \cdot {e}^{- j {ω}_{k} t} ‖}_{2}^{2} + {‖ f ( t ) - {\sum }_{k} {μ}_{k} ( t ) ‖}_{2}^{2} + 〈 λ ( t ) , f ( t ) - {\sum }_{k} {μ}_{k} ( t ) 〉$$
wherein [symbol] represents a target function for optimization, comprising the penalty coefficient and the Lagrange multiplier; λ(t) represents the Lagrange multiplier, configured to ensure that a sum of the input signal and the mode functions is equal to an original signal; f(t)−Σₖμₖ(t) represents a residual of the sum of the input signal and the mode functions, representing whether the input signal minus a combination of all of the mode functions is equal to zero; and λ(t), f(t)−Σₖμₖ(t) represents an inner product of the Lagrange multiplier and the residual, configured to ensure satisfaction of constraint conditions; and
perform iterative solution by using an alternating direction method of multipliers, and divide the unconstrained optimization problem into two sub-optimization problems to solve an overall optimal solution, in response to satisfying a convergence condition or reaching a maximum number of iterations, stop the iteration solution, and complete the signal decomposition.

2. The non-contact real-time monitoring system of physiological signs based on millimeter-wave radar as claimed in claim 1, wherein each of the electromagnetic wave signals continuously transmitted by the millimeter-wave radar is expressed as follows:
$${S}_{T} ( t ) = {A}_{T} cos ( 2 π {f}_{c} t + \frac{π B}{{T}_{c}} {t}^{2} + φ ( t ) )$$
wherein S_{T}(t) represents an electromagnetic wave signal continuously transmitted by the millimeter-wave radar, f_{c} represents an initial frequency, B represents a frequency modulation bandwidth, T_{c} represents a frequency modulation cycle, cos represents a cosine function, π represents a pi, A_{T} represents an amplitude, t represents a time, and φ(t) represents an initial phase.

3. The non-contact real-time monitoring system of physiological signs based on millimeter-wave radar as claimed in claim 1, wherein the intermediate frequency signal is expressed as follows:
$${S}_{IF} ( t ) = {A}_{T} {e}^{4 π \frac{BR ( t )}{{cT}_{c}} + 4 π \frac{{f}_{c} R ( t )}{c}}$$
wherein S_{IF}(t) represents the intermediate frequency signal, f_{c} represents an initial frequency, B represents a frequency modulation bandwidth, T_{c} represents a frequency modulation cycle, e represents a natural constant, π represents a pi, R(t) represents a function of distance changing over time, A_{T} represents an amplitude of the intermediate frequency signal, t represents a time, and c represents a speed of light.

4. The non-contact real-time monitoring system of physiological signs based on millimeter-wave radar as claimed in claim 1, wherein the clutter suppression module is further configured to estimate a degree of static clutter interference in environment by calculating an average of signals on each of range cells in a certain period, and a formula of the estimate is expressed as follows:
$$C [ n ] = \frac{1}{N} {\sum }_{m = 1}^{N} R [ n , m ]$$
wherein C[n] represents an average clutter estimate in a time window, m represents a range cell, and R[n,m] represents a radar echo signal strength received at a time point n and the range cell m.

5. The non-contact real-time monitoring system of physiological signs based on millimeter-wave radar as claimed in claim 1, wherein the target identification module is further configured to:
classify the range spectrum data into a plurality of windows each comprising data to be processed, select one of range cells from each of the plurality of windows as a center one by one, with Nᵣ/2 range cells in front and behind, and exclude a middle protection range cell to form a reference window Wᵢ;
sort values in the reference window Wᵢ in an ascending order;
calculate a threshold multiplier T, and select a kₒₛ^{th} largest value in the reference window Wᵢ as a local estimated background noise level based on a false alarm probability P_{FA} and a window size Nᵣ; and
calculate a detection threshold, in response to a test cell value of one of the range cells greater than the detection threshold, mark the one of the range cells as the target; or in response to a test unit value of one of the range cells smaller than or equal to the detection threshold, and mark the one of the range cells as a non-target.

6. The non-contact real-time monitoring system of physiological signs based on millimeter-wave radar as claimed in claim 1, wherein the range cell determination module is further configured to:
determine a range cell m₁ with a maximum reflected power by the range spectrum data after non-coherent pulse integration;
select two range cells from a left side and a right side of the range cell m₁ respectively to form a candidate set R={m₁−2, m₁−1, m₁, m₁+1, m₁+2};
extract phase information ϕ₁ and ϕ₂ of adjacent pulse signals x₁ and x₂ in a same frame according to range cells in the candidate set respectively;
extract respiratory signal components B₁ and B₂ in the phase information ϕ₁ and ϕ₂ through a [0.1 Hz, 1 Hz] band-pass filter respectively;
calculate Pearson correlation coefficients for the respiratory signal components B₁ and B₂, respectively; and
select a range cell R* with a maximum Pearson correlation coefficient between the respiratory signal component B₁ and B₂ as follows:
$${R}^{*} = argmax PCC ( {B}_{1}^{r} , {B}_{2}^{r} ) r ∈ R$$
wherein argmax represents a maximum operation, PCC represents the Pearson correlation coefficient, B₁^{r} represents a first pulse respiratory component, B₂^{r} represents a second pulse respiratory component, r represents an element in the candidate set of the range cells, and R represents the candidate set of the range cells.

7. The non-contact real-time monitoring system of physiological signs based on millimeter-wave radar as claimed in claim 6, wherein the range cell determination module is further configured to calculate the Pearson correlation coefficients for the respiratory signal components B₁ and B₂ through the following formula:
$$PCC ( X , Y ) = \frac{{\sum }_{i = 1}^{l e n} ( {X}_{i} - \overline{X} ) ( {Y}_{i} - \overline{Y} )}{\sqrt{{\sum }_{i = 1}^{l e n} {( {X}_{i} - \overline{X} )}^{2}} \sqrt{{\sum }_{i = 1}^{l e n} {( {Y}_{i} - \overline{Y} )}^{2}}}$$
wherein i represents an index variable, Xᵢ represents a value of a variable X corresponding to an i^{th} observation value, Yᵢ represents a value of a variable Y corresponding to the i^{th} observation value, len represents a number of the observation values, X̅ represents a mean value of the variable X, and Y̅ represents a mean value of the variable Y.

8. The non-contact real-time monitoring system of physiological signs based on millimeter-wave radar as claimed in claim 1, wherein the phase signal extraction module is further configured to:
calculate an initial phase φ[t] of the target through the following formula:
$$φ [ t ] = Arctan ( \frac{sin ( φ [ t ] )}{cos ( φ [ t ] )} ) - \frac{π}{2} < φ [ t ] < \frac{π}{2}$$
perform phase unwrapping to obtain an unwrapped signal sample by sample, determine whether a phase difference between adjacent samples φ[t] and φ[t+1] of the target range cell exceeds a threshold π, in response to the phase difference greater than π, subtract 2π; or in response to the phase difference not greater than π, add 2π;
perform first-order differential processing on the unwrapped signal to extract a relative phase variation between the adjacent samples to thereby obtain a signal after first-order differential processing; and
perform smooth filtering processing on the signal after first-order differential processing.
